SEQUENCE LISTING (8 sequences)

SEQ ID NO: 1 - DNA, 1971 bp, artificial
  recombinant B cell receptor specific for factor VIII
atggaaaccccagctcagcttctcttcctcctgctactctggctcccagataccaccggagaaattgcgttgacgcagtctccaggcaccctgtctttgtctccaggggaaagagccaccctctcctgcagggccagtcagagttttagcagcagctacttagcctggtatcagcagaaacctggccaggctcccaggctcctcatctatggtgcatccaccagggccactggcatcccagacaggttcagtggcagtgggtctgggacagacttcactctcaccatcagcagactggagcctgaagattttgcagtgtattactgtcagaagtatggtacgtcagcgatcaccttcgggcaagggacacgactggagattaaaggtggaggcggttcaggcggaggtggctctggcggtggcggatcgcaggtccaactggtacagtctggggctgaggtgaagaagcctggggcctcagtgaaggtctcctgcaaggtttccggatacaccctcactgaattacccgtgcactgggtgcgacaggctcctggaaaagggcttgagtgggtgggaagttttgatcctgaaagtggagaatcaatctacgcacgggagttccagggcagcgtcaccatgaccgcggacacatctacagacatagcctacatggagctgagcagcctgagatctgacgacacggccgtgtattactgtgcagtccctgaccctgatgcttttgatatctggggccaagggacaatggtcaccgtctcttcagcctccaccaagggcccatcggtcttccccctggcgccctgctccaggagcacctccgagagcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctggagctgcaactggaggagagctgtgcggaggcgcaggacggggagctggacgggctgtggacgaccatcaccatcttcatcacactcttcctgctaagcgtgtgctacagtgccaccgtcaccttcttcaaggtgaagtggatcttctcctcagtggtggacctgaagcagaccatcgtccccgactacaggaacatgataaggcagggggcctag

SEQ ID NO: 2 - protein, 656 aa, artificial
  recombinant B cell receptor specific for factor VIII
METPAQLLFLLLLWLPDTTGEIALTQSPGTLSLSPGERATLSCRASQSFSSSYLAWYQQKPGQAPRLLIYGASTRATGIPDRFSGSGSGTDFTLTISRLEPEDFAVYYCQKYGTSAITFGQGTRLEIKGGGGSGGGGSGGGGSQVQLVQSGAEVKKPGASVKVSCKVSGYTLTELPVHWVRQAPGKGLEWVGSFDPESGESIYAREFQGSVTMTADTSTDIAYMELSSLRSDDTAVYYCAVPDPDAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLELQLEESCAEAQDGELDGLWTTITIFITLFLLSVCYSATVTFFKVKWIFSSVVDLKQTIVPDYRNMIRQGA

SEQ ID NO: 3 - protein, 12 aa, artificial
  variable light chain CDR1
RASQSFSSSYLA

SEQ ID NO: 4 - protein, 7 aa, artificial
  variable light chain CDR2
GASTRAT

SEQ ID NO: 5 - protein, 9 aa, artificial
  variable light chain CDR3
QKYGTSAIT

SEQ ID NO: 6 - protein, 10 aa, artificial
  variable heavy chain CDR1
GYTLTELPVH

SEQ ID NO: 7 - protein, 17 aa, artificial
  variable heavy chain CDR2
SFDPESGESIYAREFQG

SEQ ID NO: 8 - protein, 8 aa, artificial
  variable heavy chain CDR3
PDPDAFDI

(19) United States
(12) Patent Application Publication
(10) Pub. No.: US 2011/0010786 A1
(43) Pub. Date: Jan. 13, 2011

(54) TRANSGENIC ANIMAL EXPRESSING B CELL ANTIGEN RECEPTOR

(76) Inventor: Jean-Marie Saint-Remy, Grez-Doiceau (BE)

Correspondence Address: NIXON & VANDERHYE, PC, 901 NORTH GLEBE ROAD, 11TH FLOOR, ARLINGTON, VA, 22203, US

(21) Appl. No.: 12/833,287

(22) Filed: Jul. 9, 2010

Related U.S. Application Data
(60) Provisional application No. 61/224,659, filed on Jul. 10, 2009.

(51) Int. Cl. A01K 67/027; C12N 5/10; C12N 15/63

(52) U.S. Cl. 800/18; 800/14; 435/325; 435/320.1

(57) ABSTRACT

The invention relates to a non-human transgenic mammal containing in its genome a DNA construct expressing a B cell antigen receptor specific for factor VIII of the coagulation pathway.

This application claims the benefit of U.S. Provisional Application No. 61/224,659, filed Jul. 10, 2009, the entire contents of which is hereby incorporated by reference in this application.

### FIELD OF INVENTION

The present invention relates to mouse strains expressing B lymphocytes carrying a human B cell antigen receptor specific for coagulation factor VIII and their use for evaluating the immunogenicity of factor VIII and for designing methods to reduce such immunogenicity and induce tolerance to factor VIII.

### BACKGROUND OF THE INVENTION

Hemophilia A is an X-linked disorder characterized by the absence or insufficient amount of functional factor VIII. This deficiency affects 1 in 10,000 males and can result in uncontrolled bleeding in joints, muscles and soft tissues. The coagulation pathway can be restored by administration of FVIII concentrates prepared from plasma or produced by recombinant cDNA technology.

The human FVIII gene has been isolated and expressed in mammalian cells, as reported by various authors, including Wood et al. in Nature (1984) 312:330-337 and the amino-acid sequence was deduced from cDNA. U.S. Pat. No. 4,965,199 discloses a recombinant DNA method for producing FVIII in mammalian host cells and purification of human FVIII. The human FVIII detailed structure has been extensively investigated. The cDNA nucleotide sequence encoding human FVIII and predicted amino-acid sequence have been disclosed for instance in U.S. Pat. No. 5,663,060. In a FVIII molecule, a domain may be defined as a continuous sequence of amino-acids that is defined by internal amino-acid sequence homology and sites of proteolytic cleavage by a suitable protease such as thrombin, as explained in more detail below. Human recombinant FVIII may be produced by genetic recombination in mammalian cells such as CHO (Chinese Hamster Ovary) cells, BHK (Baby Hamster Kidney) cells or other equivalent cells.

Pratt et al. (1999, Nature 402:439-42) disclose the detailed structure of the carboxy-terminal C2 domain of human FVIII, which contains sites that are essential for its binding to von Willebrand factor (vWF) and to negatively charged phospholipid surfaces. This structure, which reveals a beta-sandwich core from which two beta-turns and a loop display a group of solvent-exposed hydrophobic residues, partly explains mutations in the C2 region that lead to bleeding disorders in hemophilia A. According to Gale et al. (2000, Thromb. Haemost. 83:78-85), of the at least 250 missense mutations that cause FVIII deficiency and hemophilia A, 34 are in the C domains.

Patients suffering from hemophilia A present bleedings, which are either spontaneous in the severe form of the disease, or occur after trauma in the mild/moderate forms. Hemophilia A patients are usually treated by replacement therapy, which consists in infusing human FVIII either purified from pools of donor plasma, or obtained by cDNA recombination technology. The majority of the patients are immunologically unresponsive to these infusions, but for yet unclear reasons, 25% of them mount an IgG immune response towards FVIII, which can result in complete inhibition of the procoagulant activity of infused FVIII (Briët E et al. in (1994) Throm. Haemost.; 72: 162-164; Ehrenforth S et al. in (1992) Lancet; 339:594). Such specific IgG, which belong to the IgG 1, 2, 4 subclasses, are called FVIII inhibitors. Published studies have demonstrated that the anti-FVIII immune response is polyclonal, and primarily directed towards the A2, A3 and C2 domains (Scandella D et al. in (1989) Blood; 74: 1618-1626; Gilles J G et al. in (1993) Blood; 82: 2452-2461).

Recent studies using human monoclonal antibodies derived from the peripheral memory B cell repertoire of inhibitor patients indicated that important epitopes are also located on the C1 domain (Jacquemin M et al. in (2000) Blood 95:156-163). The mechanism by which anti-FVIII antibodies interfere with the function of FVIII are numerous, including proteolytic cleavage of FVIII and interaction with different partners such as vWF, phospholipids (PL), FIX, FXa or APC. Most of these mechanisms are now well described in studies using mouse or human anti-FVIII antibodies. Thus, antibodies can reduce the rate at which FVIII is activated by either binding to a proteolytic cleavage site or by inducing a 3D conformational change in FVIII that renders it less amenable to proteolysis. Antibodies interfering with the binding of vWF to FVIII appear to be very efficient as inhibitors, as shown in recent studies using human monoclonal antibodies directed towards the C2 domain, which is one of the major vWF binding sites (Jacquemin M et al in (1998) Blood; 92:496-501).

Suppressing the production of inhibitors and establishing a state of immune unresponsiveness to FVIII remains a major goal. The medical community is, however, far from reaching these goals, due basically to the limited understanding of the mechanisms underlying specific antibody production and regulation. Presently, to control such an immune response, several treatments are used including bypassing agents such as desmopressin (DDAVP), agents promoting coagulation such as prothrombin complex concentrates (PCC) or activated PCC, recombinant FVIIa, plasmapheresis and infusions of large or intermediate doses of FVIII (200-300 IU/kg body weight or 25-50 IU/kg body weight, respectively). However, none of these methods are satisfactory and they are all very costly.

The lack of treatment for the eradication of antibodies towards FVIII, and in particular to eliminate antibodies inhibiting the function of FVIII (so-called inhibitors) roots in the insufficiency of our knowledge about the mechanisms eliciting and sustaining the production of such antibodies. Animal models are required to elucidate such mechanisms and thereby to design novel forms of therapy to eliminate anti-FVIII antibodies. Strains of mice made deficient in FVIII have been created (Bi L. et al., (1995) Nature Genetics; 10:119-121) by knocking down expression of either exon 16 or exon 17, thereby aborting the production of a functional protein. Crossing between the initial mouse strain and strains belonging to alternative genetic backgrounds has made FVIII knocked-out (KO) mice available in a number of genetic environments. More recently, a mouse strain was created expressing a FVIII molecule carrying a mutation within the A2 domain that is commonly observed in hemophilia A patients. In addition, the crossing of FVIII-KO mice with mice rendered transgenic for a specific allele of class II major histocompatibility complexes of human origin has generated a “humanized” model of hemophilia A mice. All these models are helpful to examine some of he mechanisms by which anti-FVIII antibodies are produced, though extrapolation to the human situation remains difficult.

Hemophilia A patients lack expression of a functional FVIII molecule. As such, patients exposed to FVIII for therapeutic purposes consider FVIII as a foreign protein and mount a specific immune response to FVIII. There is therefore more of a need to understand how the production of antibodies is elicited, or in other words, how specific B cells are transformed into antibody-producing cells, than to decipher all the mechanisms by which FVIII is recognized by the immune system leading to recruitment of immunocompetent cells such as antigen-presenting cells and T cells.

### SUMMARY OF THE INVENTION

The present invention relates to transgenic animal models carrying a transgene expressing a B cell antigen receptor with specificity for FVIII. These animal models are useful to evaluate at the specific B cell level how B cells are activated after FVIII exposure, the fate and location of such B cells, their capacity to establish a memory repertoire and, based on this knowledge, to evaluate methods by which it is possible to abort B cell activation and differentiation into antibody-producing cells for the design of novel forms of therapies of use to hemophilia A patients.

In addition, B cells can be used as antigen-presenting cells for activation of T cells recognizing cognate peptides bound to major histocompatibility complex determinants. A further purpose of the B cell transgenic receptor is therefore to identify T cells for the evaluation of the mechanisms by which they are recruited, selected, activated and/or suppressed factor VIII specific T cells, and to evaluate methods by which such T cells could be prevented from activation for the design of novel forms of therapies of use in hemophilia A patients.

One aspect of the present invention relates to animal strains for use in the identification of the mechanisms by which exposure to FVIII elicits activation and expansion of B lymphocytes and their subsequent differentiation into antibody-producing cells.

Another aspect of the present invention relates to cells expressing a transgene representing a B cell antigen receptor (BCR) obtained from a human source for in vitro analysis. This allows the testing of methods to alter surface receptors expression and recognition, signaling pathways, transcriptome and epigenetic modifications induced by surface exposure to antigen and derivatives or to specific ligands such as anti-idiotypic antibodies and derivatives.

A further aspect of the present invention relates to cells expressing a transgene representing a BCR obtained from a human source for transfer to an animal for the evaluation of cell mobility and fate after transfer to an animal.

One aspect of the invention relates to non-human transgenic mammal containing in its genome a DNA construct expressing a B cell antigen receptor specific for factor VIII of the coagulation pathway. Typical animals in this context are non-human primates (e.g. baboons) and pigs and rodents, in particular mice In a particular embodiment the B cell antigen receptor is a human B cell receptor. More particularly, the B cell antigen receptor is specific for the C2 domain of Factor VIII.

In particular embodiments, the DNA construct comprises a DNA sequence of a human D segment gene, a human J segment gene and a first human constant region gene, wherein said DNA fragment is operably linked to at least one human V segment gene. More particularly, the DNA fragment is further operable linked to a second constant region gene, said second constant region gene comprising a human switch region, human CH1, C hinge, CH2 and CH3 exons and human membrane exons. In a particular embodiment, the DNA construct comprises the DNA sequence represented by SEQ ID NO: 1, or a DNA sequence having at least 90%, 95, 97, or 99 sequence identity therewith, with the proviso that the protein encoded by such sequence has Factor VIII binding properties.

Sequences encoding proteins with Factor VIII binding properties comprise for example sequences of the CDR regions of the variable light chain depicted in SEQ ID NO 3 to 5 and the CDR regions of the variable heavy chain depicted in SEQ ID NO 6 to 8.

Alternatively the DNA construct expressing B cell antigen receptor specific for factor VIII is based on the sequence of other Factor VIII inhibitory proteins as for example Krix-1 disclosed in PCT WO01/04269.

In addition to the transgene described above this non-human transgenic mammal may further have a reduced Factor VIII activity, for example a transgenic animal lacking parts of the factor VIII gene or carrying mutations in the Factor VIII gene.

Alternatively, In addition to the transgene described above this non-human transgenic mammal may further have a reduced activity of coagulation factors other than Factor VIII.

Such animals can be generated by crossing the transgenic animal of the present invention with other transgenic animals with deficiencies in the blood coagulation pathway.

An particular embodiments the non-human transgenic mammal further lacks the expression of receptors of innate immunity.

Another aspect of the present invention relates to the use of a non-human transgenic mammal as described above as a model for evaluating the immunogenicity of factor VIII.

Another aspect of the present invention relates to the use of a non-human transgenic mammal as described above as a model for evaluating the inflammatory properties of factor VIII.

Another aspect of the invention relates to an isolated population of mammalian cells containing in its genome a DNA construct which expresses a B cell antigen receptor specific for factor VIII of the coagulation pathway.

Particular embodiments relate to a population of cells which is isolated from a non-human animal as described above.

Particular embodiments relate to a population of such cells which is a population of B lymphocytes.

Another aspect of the present invention relates to a DNA construct for the expression of a B cell antigen receptor specific for factor VIII of the coagulation pathway.

Particular embodiments relate to a construct for the expression a human B cell receptor.

Particular embodiments relate to a construct wherein said B cell receptor is specific for the C2 domain of Factor VIII.

Particular embodiments relate to a construct comprising a DNA sequence of a human D segment gene, a human J segment gene and a first human constant region gene, wherein said DNA fragment is operably linked to at least one human V segment gene.

Particular embodiments relate to a construct wherein the DNA fragment further is operably linked to a second constant region gene, said second constant region gene comprising a human switch region, human CH1, C hinge, CH2 and CH3 exons and human membrane exons.

Particular embodiments relate to SEQ ID NO: 1, or a DNA sequence having at least 90%, 95, 97, or 99 sequence identity therewith, with the proviso that the protein encoded by such sequence has Factor VIII binding properties.

Sequences encoding proteins with Factor VIII binding properties comprise for example sequences of the CDR regions of the variable light chain depicted in SEQ ID NO 3 to 5 and the CDR regions of the variable heavy chain depicted in SEQ ID NO 6 to 8.

Alternatively the DNA construct expressing B cell antigen receptor specific for factor VIII is based on the sequence of other Factor VIII inhibitory proteins as for example Krix-1 disclosed in PCT WO01/04269.

A further aspect of the present invention relates to the use of an isolated DNA construct as described for the generation of transgenic non-human mammals.

A further aspect of the present invention relates to the use of the transgenic non human animal described above or the population of cells described above, for activating and subsequently identifying T cells.

In summary, the present invention relates to mouse strains transgenic for a B cell receptor specific to factor VIII of the coagulation pathway, cells derived from such strains and their use to evaluate immune and inflammatory properties of factor VIII.

### FIGURE LEGENDS

FIG. 1 shows a schematic version of an embodiment of a DNA construct for generation a transgenic mouse. V2-2C11scFv-Fc-TM transgene construct: a) Bo2C11 scFv link to its heavy chain constant and transmembrane (TM) domains inserted downstream of Ei(intronic enhancer) and pVH (promoter). b) the final transgene vector was generated by cloning the EμpVHBO2C11scFv into a second vector between isolator (I;II) and LCR (locus control region)

FIG. 2 shows genotyping performed by PCR across 2C11scFv-Fc-TM cDNA by using specific genotyping primers. Samples of mice comprising the transgene are indicated with as asterisk. Controls of the DNA construct (C), wild type mice (W) and water (H) are indicated.

FIG. 3 shows a FACS analysis using either labeled donkey IgG to human IgG or an anti-idiotypic antibody to B02C11 showing that 1-3% of peripheral B cells and 15-40% of spleen and bone marrow B cells carry the transgene.

FIG. 4 shows the effect of FVIII I.V. injection (10 UI/mouse) in transgenic mice as increases in the number of Tg BCR-2C11 cells in the spleen (left panel) and in the bone marrow (right panel) as detected by FACS.

FIG. 5 shows that the I.V. injection of FVIII increased the % of Tg-BCR 2C11(+) in the CD19(+) spleen cell population as measured by FACS. (Detection of CR-2C11 on CD19 spleen cells after FVIII stimulation)

FIG. 6 shows that I.V. injection of FVIII or of anti-Id Ab 14C12 increased the % of Tg-BCR 2C11(+) in the CD19(+) bone marrow cell population. (Detection of BCR-2C11 on CD19 bone marrow cells after different Ag stimulations (recFVIII, 14C12, ScFv).

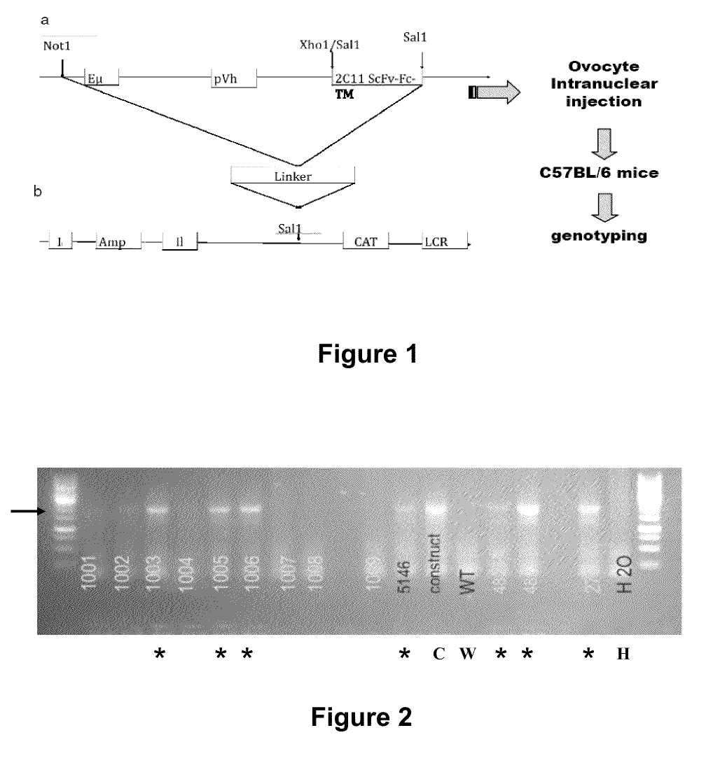

### DETAILED DESCRIPTION OF THE INVENTION

### Definitions

“B cell antigen receptor (BCR)” also known as “membrane or surface immunoglobulin” relates to a membrane-bound form of an antibody It is part of the B cell receptor (BCR), which allows a B cell to detect when a specific antigen is present in the body and triggers B cell activation.
“Hemophilia A” is an X-linked disorder characterized by the absence or insufficient amount of functional factor VIII, a 330 kD glycoprotein molecule produced by the liver as a single polypeptide chain of 2332 amino acids. This deficiency affects 1 in 10,000 males and can result in uncontrolled bleeding in joints, muscles and soft tissues. Patients affected by the severe form of the disease (FVIII activity lower than 1% of normal level) suffer from spontaneous bleedings. Patients with corresponding FVIII activity from 1 to 5%, or higher than 5% are defined as moderate or mild hemophilia A, respectively, and suffer from limited bleeding occurring after minor trauma or surgery.
“FVIII (factor VIII)” is a cofactor of the intrinsic pathway of the coagulation cascade, which acts by increasing the proteolytic activity of activated factor IX over factor X, in the so-called tenase complex formation.
“Domain” in the present invention refers to a part of Factor VIII. FVIII proteins has been described to consist of different domains, which for the human amino-acid sequence correspond to: A1, residues 1-372; A2, residues 373-740; B, residues 741-1648; A3, residues 1690-2019; C1, residues 2020-2172; C2, residues 2173-2332. The remaining sequence, residues 1649-1689, is usually referred to as the FVIII light chain activation peptide. FVIII is produced as a single polypeptide chain which, upon processing within the cell, is rapidly cleaved after secretion to form a heterodimer made of a heavy chain containing the A1, A2 and B domains and a light chain made of the A3-C1-C2 domains, according to Kaufman et al. (1988, J. Biol. Chem. 263:6352-6362). The two chains are non-covalently bound by divalent cations. Both the single-chain polypeptide and the heterodimer circulate in plasma as inactive precursors, as taught by Ganz et al. (1988, Eur. J. Biochem. 170:521-528). Activation of factor VIII in plasma initiates by thrombin cleavage between the A2 and B domains, which releases the B domain and results in a heavy chain consisting of the A1 and A2 domains, according to Eaton et al. (1986, Biochemistry 25:505-5121).
“FVIII inhibitor” refers to specific IgGs, which belong to the IgG 1, 2, 4 subclasses. These molecules mount an IgG immune response towards FVIII, which can result in complete inhibition of the procoagulant activity of infused FVIII (Briët E et al. in (1994) Throm. Haemost.; 72: 162-164; Ehrenforth S et al. in (1992) Lancet; 339:594). The mechanism by which such anti-FVIII antibodies interfere with the function of FVIII are numerous, including proteolytic cleavage of FVIII and interaction with different partners such as vWF (von Willebrand Factor), phospholipids (PL), FIX, FXa or APC.
“Transgene” in the present invention refers to a nucleic acid sequence that has been introduced into a cell by way of human intervention. A transgene could be partly or entirely heterologous, i.e., foreign, to the transgenic animal or cell into which it is introduced. A transgene can include one or more transcriptional regulatory sequences and any other nucleic acid, such as introns, that may be necessary for optimal expression of a selected nucleic acid.
“Transgenic animal” relates to any non-naturally occurring non-human mammal in which one or more of the cells of the animal contain heterologous nucleic acid encoding a B cell antigenic receptor specific to Factor VIII, that has been introduced by way of human intervention, such as by transgenic techniques well known in the art.

A transgene nucleic acid is introduced into the cell, directly or indirectly by introduction into a precursor of the cell, by way of deliberate genetic manipulation, such as by microinjection or by infection with a recombinant virus. A transgene may be integrated within a chromosome, or it may be extrachromosomally replicating DNA.

Methods for generating non-human transgenic animals of the present invention, are well known in the art. Laboratory manuals for this purpose include “Gene Targeting: A Practical Approach,” Joyner, ed., Oxford University Press, Inc. (2000); Robertson, E. J. ed. “Teratocarcinomas and Embryonic Stem Cells, a Practical Approach”, IRL Press, Washington D.C., 1987 and “Manipulating the Mouse Embryo’, Hogan eds. (Cold Spring Harbor Laboratory Press, Cold Spring Harbor, 1986).

“Animal” in the present invention relates to mammals, and for the purpose of transgenic animals to non-human animals. Particular mammals in the context of the present invention or rodent, such as rats and mice (e.g. mus musculus and other laboratory mice). Other particular mammals in the context of the study of bleeding disorders or non-human primates such as baboons.

The human monoclonal antibody BO2C11 (hereafter referred to as BO2C11) is a FVIII-specific antibody derived from the natural repertoire of a patient with inhibitor (Jacquemin M G, et al. in (1998) Blood 92: 496-506). Antibody BO2C11 inhibits the binding of FVIII to both vWF and PL. This antibody is representative of a major class of human inhibitory antibodies. Its mechanism of action is commonly encountered in patients with inhibitor and C2-specific antibodies are the most frequently observed inhibitory antibodies. Antibody BO2C11 was fully characterized as reported in Jacquemin et al. (Blood, 92: 496-506, 1998). The sequence of the heavy and light variable chain have been submitted to Genbank with Accession number AJ224083 and AJ224084, respectively. The CDR regions of this antibody are represented in FIGS. 6 and 7 of PCT WO01/04269. BO2C11 is an IgG4k antibody carrying a VH segment of the DP-5 subfamily (part of VH-1 family) and a J segment homologous to JH3b. Light chain sequencing identified VL as a VkIII and a J segment as a Jk5. Specificity is for the C2 domain of factor VIII, more precisely the PL binding sites of the C2 domains, namely 2 beta-turn regions corresponding to residues 2250 to 2253 and 2197 to 2203, respectively, with, in addition, a loop containing residues 2222 and 2223. Complete description of such epitope can be found in Spiegel P C et al. (Blood, 98: 13-19, 2001), which reports the crystal structure of BO2C11 together with the C2 domain of factor VIII. Additional information on epitope mapping was obtained by the use of mutant C2 domain in which single amino acid residues were substituted by alanine, indicating that residue Ala2201 was critical for antibody binding. Sequencing of the variable parts of BO2C11 showed mutations in the complementarity-determining regions (CDRs), indicating that the antibody had undergone affinity maturation. Measurement of antibody affinity for factor VIII using Surface Plasmon Resonance provided the following values: kₐₛₛ 2×10⁵ mol/L⁻¹s⁻¹, kdiss≦1×10-5s-1

BO2C11 cross-reacts with mouse FVIII with an affinity similar to that for human factor VIII, but not to porcine or canine factor VIII.

### EXAMPLES

### 1 Construction of a Transgenic Mouse

### 1.1 Source of the Transgene

A transgene is derived from a B cell clone obtained from the peripheral blood of a hemophilia A patient (BO) with a high titer of inhibitor. The method used is described in full in Jacquemin et al. (Blood, 92: 496-506, 1998). Briefly, peripheral mononucleated cells were obtained by venipuncture and a purified preparation of memory B cells was obtained by sorting on magnetic beads. Memory B cells were then transformed by a combination of Epstein-Barr virus transformation and CD40 activation in the presence of cytokines. Cloning was carried out by limiting dilutions. The sequence of VH and VL segments as well as the sequences of the constant parts of both the heavy and light chains were obtained by reverse transcription and PCR amplification. Such sequences were then cloned in vectors for transgenesis (see below).

### 1.2. Description of Constructs and Transgenesis

### Construction of Vectors for Transgenesis

An artificial antibody made of the Bo2C11 scFv (single chain variable fragment) linked to its heavy chain constant and transmembrane (TM) domains (2C11scfv-Fc-TM). Briefly, mRNA from BO2C11 hybridoma was isolated using the Quick Prep Micro mRNA Purification Kit (Amersham Pharmacia Biotech, Uppsala, Sweden). cDNA was synthesized with first cDNA synthesis kit (Amersham Pharmacia Biotech). The heavy and light chain (VL) variable regions (VH and VL, respectively), and the Fc and TM domains were amplified by PCR using specific primers. All cDNA fragments were assembled and cloned into the pGEM-Teasy vector (Promega, Madison, WT), in accordance with the instructions of the manufacturer. The assembled cDNA fragment is represented below as SEQ ID NO:1 (cDNA sequence) and SEQ ID NO:2 (protein sequence). The CDR regions are indicated in this sequence and also represented individually below the sequence.

| | |
|---|---|
| atggaaaccccagctcagcttctcttcctcctgctactctggctcccagataccaccgga | 60 |
| M E T P A Q L L F L L L L W L P D T T G | |
| gaaattgcgttgacgcagtctccaggcaccctgtctttgtctccaggggaaagagccacc | 120 |
| E I A L T Q S P G T L S L S P G E R A T | |
| ctctcctgcagggccagtcagagttttagcagcagctacttagcctggtatcagcagaaa | 180 |
| L S C R A S Q S F S S S Y L A W Y Q Q K | |
| ---------CDR1 VL-[SEQ ID NO 3]---- | |
| cctggccaggctcccaggctcctcatctatggtgcatccaccagggccactggcatccca | 240 |
| P G Q A P R L L I Y G A S T R A T G I P | |
| -CDR2 VL[SEQ ID NO 4]- | |
| gacaggttcagtggcagtgggtctgggacagacttcactctcaccatcagcagactggag | 300 |
| D R F S G S G S G T D F T L T I S R L E | |
| cctgaagattttgcagtgtattactgtcagaagtatggtacgtcagcgatcaccttcggg | 360 |
| P E D F A V Y Y C Q K Y G T S A I T F G | |
| -CDR 3 VL [SEQ ID NO 5]-- | |
| caagggacacgactggagattaaaggtggaggcggttcaggcggaggtggctctggcggt | 420 |
| Q G T R L E I K G G G G S G G G G S G G | |
| ggcggatcgcaggtccaactggtacagtctggggctgaggtgaagaagcctggggcctca | 480 |
| G G S Q V Q L V Q S G A E V K K P G A S | |
| gtgaaggtctcctgcaaggtttccggatacaccctcactgaattacccgtgcactgggtg | 540 |
| V K V S C K V S G Y T L T E L P V H W V | |
| ---CDR1 VH [SEQ ID NO 6]---- | |
| cgacaggctcctggaaaagggcttgagtgggtgggaagttttgatcctgaaagtggagaa | 600 |
| R Q A P G K G L E W V G S F D P E S G E | |
| ----CDR2 VH----------- | |
| tcaatctacgcacgggagttccagggcagcgtcaccatgaccgcggacacatctacagac | 660 |
| S I Y A R E F Q G S V T M T A D T S T D | |
| -------[SEQ ID NO 7]----- | |
| atagcctacatggagctgagcagcctgagatctgacgacacggccgtgtattactgtgca | 720 |
| I A Y M E L S S L R S D D T A V Y Y C A | |
| gtccctgaccctgatgcttttgatatctggggccaagggacaatggtcaccgtctcttca | 780 |
| V P D P D A F D I W G Q G T M V T V S S | |
| -CDR3 VH [SEQ ID NO 8]- | |
| gcctccaccaagggcccatcggtcttccccctggcgccctgctccaggagcacctccgag | 840 |
| A S T K G P S V F P L A P C S R S T S E | |
| agcacagcggccctgggctgcctggtcaaggactacttccccgaaccggtgacggtgtcg | 900 |
| S T A A L G C L V K D Y F P E P V T V S | |
| tggaactcaggcgccctgaccagcggcgtgcacaccttcccggctgtcctacagtcctca | 960 |
| W N S G A L T S G V H T F P A V L Q S S | |
| ggactctactccctcagcagcgtggtgaccgtgccctccagcagcttgggcacgaagacc | 1020 |
| G L Y S L S S V V T V P S S S L G T K T | |
| tacacctgcaacgtagatcacaagcccagcaacaccaaggtggacaagagagttgagtcc | 1080 |
| Y T C N V D H K P S N T K V D K R V E S | |
| aaatatggtcccccatgcccatcatgcccagcacctgagttcctggggggaccatcagtc | 1140 |
| K Y G P P C P S C P A P E F L G G P S V | |
| ttcctgttccccccaaaacccaaggacactctcatgatctcccggacccctgaggtcacg | 1200 |
| F L F P P K P K D T L M I S R T P E V T | |
| tgcgtggtggtggacgtgagccaggaagaccccgaggtccagttcaactggtacgtggat | 1260 |
| C V V V D V S Q E D P E V Q F N W Y V D | |
| ggcgtggaggtgcataatgccaagacaaagccgcgggaggagcagttcaacagcacgtac | 1320 |
| G V E V H N A K T K P R E E Q F N S T Y | |
| cgtgtggtcagcgtcctcaccgtcctgcaccaggactggctgaacggcaaggagtacaag | 1380 |
| R V V S V L T V L H Q D W L N G K E Y K | |
| tgcaaggtctccaacaaaggcctcccgtcctccatcgagaaaaccatctccaaagccaaa | 1440 |
| C K V S N K G L P S S I E K T I S K A K | |
| gggcagccccgagagccacaggtgtacaccctgcccccatcccaggaggagatgaccaag | 1500 |
| G Q P R E P Q V Y T L P P S Q E E M T K | |
| aaccaggtcagcctgacctgcctggtcaaaggcttctaccccagcgacatcgccgtggag | 1560 |
| N Q V S L T C L V K G F Y P S D I A V E | |
| tgggagagcaatgggcagccggagaacaactacaagaccacgcctcccgtgctggactcc | 1620 |
| W E S N G Q P E N N Y K T T P P V L D S | |
| gacggctccttcttcctctacagcaggctaaccgtggacaagagcaggtggcaggagggg | 1680 |
| D G S F F L Y S R L T V D K S R W Q E G | |
| aatgtcttctcatgctccgtgatgcatgaggctctgcacaaccactacacgcagaagagc | 1740 |
| N V F S C S V M H E A L H N H Y T Q K S | |
| ctctccctgtctctggagctgcaactggaggagagctgtgcggaggcgcaggacggggag | 1800 |
| L S L S L E L Q L E E S C A E A Q D G E | |
| ctggacgggctgtggacgaccatcaccatcttcatcacactcttcctgctaagcgtgtgc | 1860 |
| L D G L W T T I T I F I T L F L L S V C | |
| tacagtgccaccgtcaccttcttcaaggtgaagtggatcttctcctcagtggtggacctg | 1920 |
| Y S A T V T F F K V K W I F S S V V D L | |
| aagcagaccatcgtccccgactacaggaacatgataaggcagggggcctag | 1971 |
| K Q T I V P D Y R N M I R Q G A | |
| (linker is underlined) | |
| CDR1 VL [SEQ ID NO 3] RASQSFSSSYLA | |
| CDR2 VL [SEQ ID NO 4] GASTRAT | |
| CDR3 VL [SEQ ID NO 5] QKYGTSAIT | |
| CDR1 VH [SEQ ID NO 6] GYTLTELPVH | |
| CDR2 VH [SEQ ID NO 7] SFDPESGE SIYAREFQG | |
| CDR3 VH [SEQ ID NO 8] PDPDAFDI | |

Murine genomic fragments corresponding to pVH (variable heavy chain promoter), Eμ (intronic enhancer) and 3′ regulatory enhancer region named LCR (locus control region) were inserted into a V2 vector. The combination of the intronic and 3′ enhancers with a pVH promoter allows a cooperation between each element to ensure correct spatial and temporal expression of the gene to be expressed. The Eμ-LCR combination strictly restricts transgene expression to the B lymphocyte lineage (Cogné et al. Biochem Biophys Acta, 2003; 1642:181-190).

The cDNA encoding for the 2C11scfv-Fc-TM was amplified by PCR and assembled into the correct site, downstream of the Eμ and pVH promoter and upstream of the LCR region (V2-2C11scfv-Fc-TM).

### Transgenesis

The generation of transgenic mice was carried out by microinjection. Briefly, the linearized V2-2C11scfv-FC-TM transgene construct was microinjected into the male pronucleus of inbred C57BL/6J zygotes, using 2 ng/μl in Tris 10 mM, EDTA 0.1 mM buffer. Surviving embryos were transferred into B6C3 F1 foster mice at day 0.5, as described (Ectors et al. (2007) Differentiation 75, 256-267). Mice were maintained under specific pathogen free conditions (SPF).

### 1.3. Description of the Transgenic Mouse Strains

### Genotypic Characterization

Genomic DNA was purified from tail biopsies of founder mice at 3 weeks of age. Genotyping was performed by PCR across 2C11scfv-Fc-TM cDAN by using specific genotyping primers. The PCR reactions were incubated at 95° C. for 5′, followed by 40 cycles of 95° C. for 30 seconds, 50° C. for 30 seconds, 72° C. for 1 minute followed by 72° C. for 7 minutes.

### Phenotypic Characterization

The BO2C11-BCR transgenic C57BL/6 strain (BO2C11-Tg strain) shows normal growth, overall phenotype and fertility. Histologic assessment of lymphoid organs show normal architecture. The number and proportion of B cells and T cells are within normal range.

The percentage of B cells carrying the transgenic BCR was evaluated in blood, spleen and bone marrow and typically found in between 0.5 and 40%. Transgenic expression was observed on marginal zone (MZ) as well as on follicular B cells.

At rest, and with no previous exposure to the antigen, transgenic B cells show a surface phenotype characteristic of naïve B cells: CD80^{high}, CD86^{low}, CD40^{low}, CD45RA^{high}, ICAM^{low}, CD62L^{low}, MHC class II^{low}

After activation by in vitro exposure to FVIII, transgenic B cells showed a pattern of early signaling with phosphorylation of ITAMs, Igalpha/beta, Syk and Lyn, demonstrating the functionality of the antigen receptor.

In vivo activation of transgenic B cells by FVIII administration increases the proportion of cells expressing the transgenic receptor significantly and shows relocation of cells into the bone marrow.

The present data show that the transgenic animal model is suitable for the evaluation of B cell reactivity towards factor VIII.

### Backcrossing

The BO2C11-Tg strain was backcrossed with a C57BL/6 mouse strain carrying a FVIII exon 16 deletion (FVIII-KO), thereby creating a FVIII-KO-BO2C11-Tg strain. The phenotypic characterization of such FVIII-KO-BO2C11-Tg strain was essentially similar to the BO2C11-Tg strain.

### Example 2

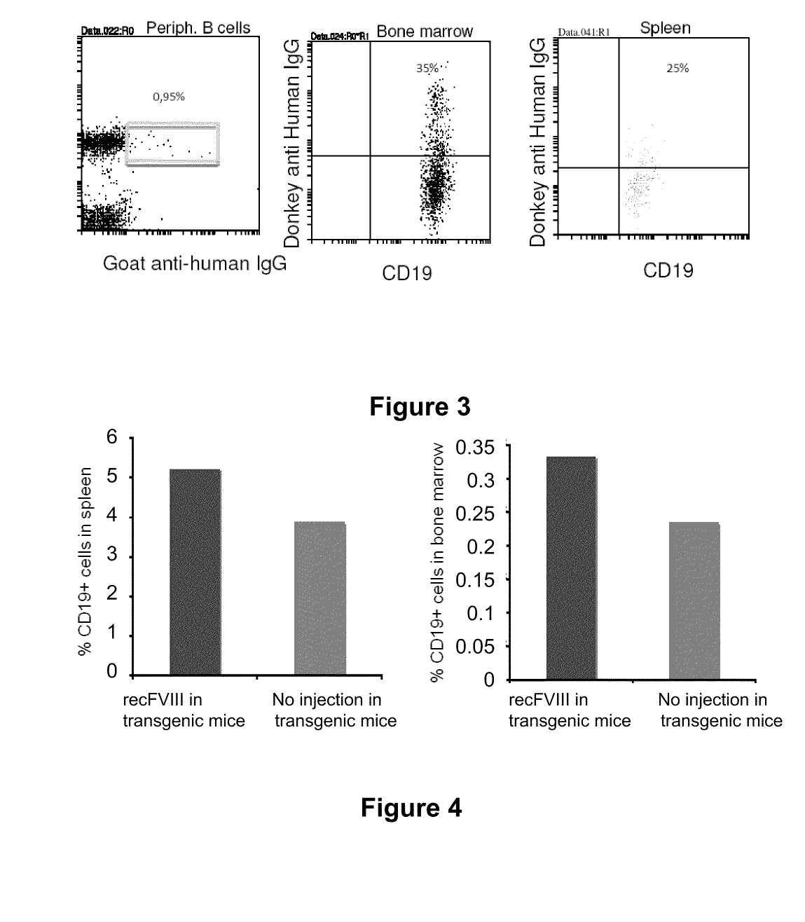

### B Cells Carrying the Transgenic Receptor (FIG. 3)

B cells were prepared from the spleen of a BCR transgenic mouse by purification using magnetic beads coated with an anti-CD19 antibody. CD19+ cells were then incubated with a fluorescent-labeled donkey anti-human IgG antibody prior to evaluating the proportion of labeled cells using a fluorescence-activated cell sorter (FACS). The figure shows representative data of such experiment with 35% transgenic B cells in the bone marrow and 29% in the spleen. In addition, peripheral blood mononucleated cells (PBMC) were prepared by density gradient centrifugation and incubated with both an anti-CD19 antibody and a goat anti-human IgG. It shows that ±1% of circulating B cells express the transgenic receptor.

FACS analysis using either labeled donkey IgG to human IgG or an anti-idiotypic antibody to BO2C11 showed that 1-3% of peripheral B cells and 15-40% of spleen and bone marrow B cells carried the transgene.

### Example 3

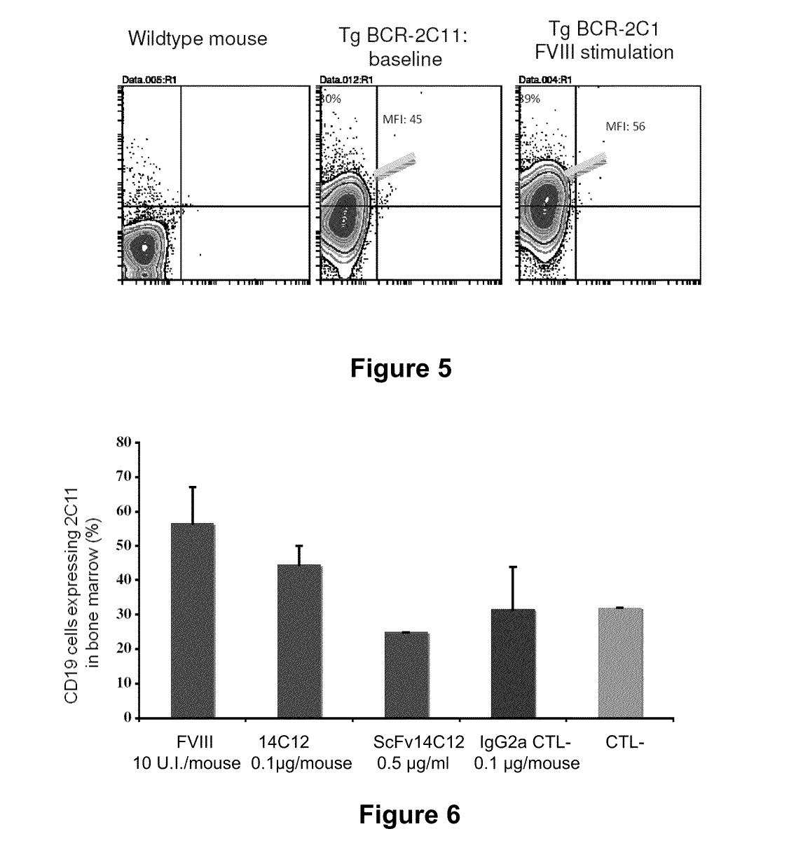

### Functionality of Transgenic B Cells (FIG. 5)

To assess the functionality of the transgenic B cell antigen receptor, 2 IU of FVIII was injected in either a wildtype C57BL/6 mouse or in a C57BL/6 BCR transgenic mouse. Three days later the mice were sacrificed together with a control transgenic mouse that was not injected with FVIII. CD19+ B cells were prepared from the spleen by magnetic bead sorting and the cells further incubated with a donkey anti-human IgG antibody to detect transgene expression. Results show that injection of FVIII significantly increased the % of B cells expressing the transgene in the spleen.

### Example 4

### Effect of FVIII on Transgenic B Cells

To evaluate in vitro the effect of FVIII on transgenic B cells, the latter were purified from spleen total B cells by magnetic depletion of non-B cells by magnetic beads. B cells were purified from wildtype syngeneic mice as a control. CD138+ plasma cells were then removed on beads coated with a specific antibody. The CD138(−) cell population was analyzed by flow cytometry. Expression levelS of surface markers such as CD80, CD62-ligand and MHC class II molecules showed cells to share characteristics of naïve B cells.

2×10⁵ CD138⁻ cells were incubated in vitro for 20 hours with recombinant FVIII (rFVIII) at different concentrations (from 0.01 to 20 IU/ml) or with the same concentrations of Von Willebrand Factor (vWF)-containing FVIII at a rFVIII/vWF molecular ratio of 1/50. CD138⁻ cells were incubated with buffer as negative control. Apoptosis was detected by staining with annexin-V and 7-AAD and compared to CD138(−) cells obtained from wildtype animals. Transgenic cells were identified using an anti-human IgG specific antibody. The results indicated that at lower or physiological concentrations of rFVIII (0.01 to 1 IU/ml), no significant apoptosis was observed. At higher rFVIII concentration (201 U/ml), significant apoptosis was observed with both transgenic and non-specific cells, identifying a potential cytotoxicity.

| | CD138(−) cells (% apoptosis) | | | | | |
|---|---|---|---|---|---|---|
| | WT C57BL/6 mice | | | Transgenic BCR 2C11 mice | | |
| | Concentration | | | | | |
| | 0.01 I.U to | | | 0.01 I.U to | | |
| | 1 I.U./ml | 10 I.U./ml | 20 I.U./ml | 1 I.U./ml | 10 I.U./ml | 20 I.U./ml |
| rec FVIII | 0% | 80% | 100% | 0% | 80% | 100% |
| rec FVIII/vWF | 0% | 80% | 100% | 0% | 80% | 100% |
| Dialysed recFVIII | 0% | 0% | 0% | 0% | 0% | 0% |

Further, in vivo data showed that rFVIII or an anti-idiotypic antibody specific to the transgenic BCR (14C12) results in significant activation of transgenic B cells.

These data confirm that the BCR transgenic model allows evaluating B cell reactivity towards FVIII.

1. A non-human transgenic mammal containing in its genome a DNA construct expressing a B cell antigen receptor specific for factor VIII of the coagulation pathway.

2. The non-human transgenic mammal according to claim 1 which is a rodent.

3. The non-human transgenic mammal according to claim 1 which is mouse.

4. The non-human transgenic mammal according to claim 1, wherein said B cell antigen receptor is a human B cell receptor.

5. The non-human transgenic mammal according to claim 1, wherein the B cell antigen receptor is specific for the C2 domain of Factor VIII.

6. The non-human transgenic mammal according to claim 1, wherein said DNA construct comprises a DNA sequence of a human D segment gene, a human J segment gene and a first human constant region gene, wherein said DNA fragment is operably linked to at least one human V segment gene.

7. The non-human transgenic mammal according to claim 6 wherein said DNA fragment further is operably linked to a second constant region gene, said second constant region gene comprising a human switch region, human CH1, C hinge, CH2 and CH3 exons and human membrane exons.

8. The non-human transgenic mammal according to claim 1 wherein said DNA construct comprises the DNA sequence represented by SEQ ID NO: 1, or a DNA sequence having at least 90% sequence identity therewith.

9. The non-human transgenic mammal according to claim 1 wherein said DNA construct comprises the sequences of the CDR regions of the variable light chain depicted in SEQ ID NO 3 to 5 and the CDR regions of the variable heavy chain depicted in SEQ ID NO 6 to 8.

10. The non-human transgenic mammal according to claim 1, which further has a reduced Factor VIII activity.

11. The non-human transgenic mammal according to claim 1 wherein said reduced Factor VIII activity is achieved by the introduction of a transgenic construct lacking parts of the factor VIII gene or a transgenic construct carrying mutations in the Factor VIII gene.

12. The non-human transgenic mammal according to claim 1, which further has a reduced activity of coagulation factors other than Factor VIII.

13. The non-human transgenic mammal according to claim 1, which further lacks the expression of receptors of innate immunity

14. Use of a non-human transgenic mammal in accordance to claim 1 as a model for evaluating the immunogenicity of factor VIII.

15. Use of a non-human transgenic mammal in accordance to claim 1 as a model for evaluating the inflammatory properties of factor VIII.

16. An isolated population of mammalian cells containing in its genome a DNA construct which expresses a B cell antigen receptor specific for factor VIII of the coagulation pathway.

17. The population of cells according to claim 16 which is isolated from a non-human animal.

18. The population of cells according to claim 16 which is a population of B lymphocytes.

19. An isolated DNA construct for the expression of a B cell antigen receptor specific for factor VIII of the coagulation pathway, wherein said B cell receptor is a human B cell receptor, wherein said B cell receptor is specific for the C2 domain of Factor VIII, and wherein said DNA construct comprises a DNA sequence of a human D segment gene, a human J segment gene and a first human constant region gene, wherein said DNA fragment is operably linked to at least one human V segment gene.

20-22. (canceled)

23. The isolated DNA construct according to claim 19, wherein said DNA fragment further is operably linked to a second constant region gene, said second constant region gene comprising a human switch region, human CH1, C hinge, CH2 and CH3 exons and human membrane exons.

24. The isolated DNA construct according to claim 19 comprising the DNA sequence represented by SEQ ID NO: 1, or a DNA sequence having at least 90% sequence identity therewith.

25. The isolated DNA construct according to claim 19 comprising the sequences of the CDR regions of the variable light chain of BO2C11 depicted in SEQ ID NO 3 to 5 and comprising the CDR regions of the variable heavy chain of BO2C11 depicted in SEQ ID NO 6 to 8.

26-27. (canceled)
